(19) United States
(12) Patent Application Publication
(10) Pub. No.: US 2013/0073019 A1
(43) Pub. Date: Mar. 21, 2013

(54) Bacterial Cellulose-Based Ice Bags and the Production Method Thereof

(71) Applicant: Chunyan Zhong, Hainan (CN)

(72) Inventor: Chunyan Zhong, Hainan (CN)

(21) Appl. No.: 13/512,307

(22) PCT Filed: Jun. 8, 2010
(86) PCT No.: PCT/CN10/01197

§ 371 Date: Aug. 22, 2012

(30) Foreign Application Priority Data
Jun. 7, 2010 (CN) 20100205354.1

(51) Int. Cl. A61F 7/10; B65B 3/04

(52) U.S. Cl. CPC A61F 7/10; B65B 3/04
USPC 607/114; 53/425; 435/252.1; 568/840; 536/56; 568/852; 252/73

(57) ABSTRACT

This invention relates to bacterial cellulose-based ice bags and their production methods thereof. Bacterial cellulose membranes used in the present invention are produced through the fermentation of cellulose-producing microorganisms. Ice bags with three sealed sides and one open side are made from water-impermeable (under room temperature and normal atmospheric pressure) bacterial cellulose membranes. The bags are filled with filler materials and then sterilized at 120 degrees C. for 30 minutes and sealed aseptically. The use of bacterial cellulose as the packaging material solves the problems associated with conventional ice bags. The advantages of the present invention include: no condensation at the bag surface, smaller temperature changes during the course of application, and longer lasting ice therapy session. In addition, bacterial cellulose membrane is safe and non-toxic, and has excellent biocompatibility, plasticity and higher mechanical strength, making it an ideal packaging material for ice bags.

### FIELD OF INVENTION

The present invention relates to bacterial cellulose-based ice bags and their production methods in the field of biological engineering.

### BACKGROUND OF INVENTION

Ice therapy is a commonly used method of physical therapy. It uses ice or ice water mixture to cover the affected area to reduce swelling, bleeding, pain, and to achieve remission or treatment of disease.

Human tissues, especially soft tissues, when injured will enter into stages of swelling, itching, fever, and pain. Physiological responses to injuries vary significantly with temperature. On the one hand, when the temperature drops to twenty degrees Celsius, the skeletal muscle of the muscle spindle reflex will be inhibited, reducing muscle tension and pain or blocking nerve conduction. As the temperature drops to less than ten degrees Celsius, the tissue's emergency relief reflex slows down and causes contraction of blood vessels near the injured cells and damaged capillaries, which in turn reduces bleeding and slows down cell metabolism. In addition it also suppresses the stimulation of nerve endings and eases inflammatory conditions, effectively relieving pain. On the other hand, low temperature will strengthen the collagen fibers (tendons, ligaments, cartilage, etc.) to reduce aggravation of injured muscle and tendon resulted from the body's own stress response, minimizing tissue damages. Early intervention with local ice treatment can effectively protect the injured tissues from harmful secondary injuries.

Ice therapy is used in the acute injuries such as surgery, sports injury, fever and other inflammatory conditions. Although the treatment is quite effective, there is still room for improvement in current ice therapy devices:

First, the conventional ice therapy for patients with fever is the application of an ice bag to the head, neck, cheek, jaw or other body parts. The most commonly used method is to use the traditional ice bags (with ice cubes and water mixture). Because the hardness and sharp edges of the ice cubes, application of the ice bag may cause discomfort to the affected area, resulting in poor contact to the tissue and thus unsatisfactory results. This is especially true when the patient is experiencing emotional distress caused by the intolerable pain.

Second, to address the drawbacks of conventional ice bags, ice packs with ethanol and propylene glycol as coolants and polyethylene, polyvinyl chloride, or polypropylene film as packaging material have been developed. These types of ice packs are easier to apply and have better stability when affixed to the affected areas. However, these types of ice packs are prone to condensation and formation of water droplets on the polyethylene, polyvinyl chloride, polypropylene films and other packaging materials. The water droplets formed on outside surface not only can cause inconvenience to the patient, but it also can result in uneven cooling or dampness to local wounds, causing infection or other serious consequences.

Third, the heat transfer coefficients of conventional ice pack packaging materials are high, causing dramatically temperature changes upon application due to rapid heat exchange with body heat. In reality the time of application is usually less than 20 minutes each time. Therefore typical cold therapy is done with multiple short sessions separated by at least 30 minutes of rest between sessions. More importantly, the huge temperature difference between start and end of application may cause intense contraction of blood vessels, leading to frostbite or, more seriously, tissue necrosis or even amputation.

### DESCRIPTION

The purpose of the present invention is to address the deficiency of existing technologies used in conventional ice bag materials. The invention teaches the use of bacterial cellulose membranes as ice bag packaging material and the production method of such ice bags to address the drawbacks such as condensation problem and huge temperature changes before and after application found in conventional ice bag packaging materials. The current invention has the advantages of being able to provide a long-lasting cooling effect and being a safe, nontoxic material with high biocompatibility, plasticity, and mechanical strength, making it an ideal material for ice bags intended for physical therapy.

The technical principles used in the invention:

Designing a bacterial cellulose membrane to match the shapes and structural features of a patient's legions and ultimately producing a new bacterial cellulose-based ice bag.

Bacterial cellulose is characterized by the following properties:
1. Bacterial cellulose is a natural material with an excellent biocompatibility that allows it to be used inside and on the surface of body without causing allergies, immune rejection and other adverse reactions.
2. Bacterial cellulose has a high Young's modulus (15 GPa, or up to 30 GPa after heat treatment, equivalent to that of aluminum), making it resistant to high shear and tensile forces, and allowing it to withstand strong external force;
3. Bacterial cellulose is a biopolymer composed of D-glucose units linked by β-1,4 glycosidic bonds. It has a strong hydrophilicity and high water-retention capacity (water retention ratio of 1:50, and up to 1:700 with special treatments), and thus prevent condensation on the ice bag surface from occurring.
4. Bacterial cellulose film has a high plasticity which would allow the ice bag to fit the contour of affected area.
5. It is biodegradable.

To achieve these objectives, the present invention employs the following in the technologies:

The invention relates to bacterial cellulose-based ice bags and production methods thereof. The production of ice bags involves selecting bacterial cellulose film as the packaging material, and aseptically filling and sealing the bags with appropriate thickening cooling agents hydrated in distilled water after sterilization.

The specific steps are as follows:
1. The preparation of bacterial cellulose membrane: using cellulose producing microorganism as starting culture to make the bacterial cellulose membrane through fermentation. The said microorganism is the Gluconacetobacter xylinum 323 strain.
2. Selection of bacterial cellulose membrane with appropriate shape and structural strength: choose bacterial cellulose membranes that are impermeable to water under normal temperature and pressure as packaging material. Make the ice bag (sealed on three sides with one side unsealed) according to the actual shape and size of the lesion.
3. Fill the bacterial cellulose ice bag with filler materials, followed by sterilization at 120 deg. C. for 30 minutes and sealing the bag aseptically. The said filler materials include cooling agent, a mixture of cooling agent and thickener, a mixture of cooling agent and distilled water, or a mixture of cooling agent, thickeners and distilled water. The said cooling agent is glycerol, ethanol or a mixture of glycerol and ethanol. The said thickener is methyl cellulose.

In the present invention, the production cultures include, but not limited to, Acetobacter, Agrobacterium, Rhizobium and Sarcina species.

The invention has the following advantages:
1. Prevent condensation on the surface of the ice bags. Bacterial cellulose is a polyglucan. It has a strong hydrophilicity and high water-retention capacity (water retention ratio of 1:50, and up to 1:700 with special treatments), and thus prevent condensation on the ice bag surface from occurring;
2. Bacterial cellulose has a high Young's modulus, making it resistant to high shear and tensile forces, and breakage;
3. Excellent biocompatibility. Bacterial cellulose is a natural biopolymer and it does not cause allergies, immune rejection and other adverse reactions;
4. Biodegradable. Bacterial cellulose can be degraded easily by cellulase present in the nature and therefore is environmental friendly.

### SPECIFIC IMPLEMENTATION EXAMPLES

The following non-restrictive embodiments are used to further describe the invention in detail.

### EXAMPLE 1

### Preparation of Occipital Peripheral Ice Bags

1. Preparation of packaging materials: Using the occipital peripheral area as blueprint, make a rectangular ice bag (20 cm (l)×8 cm (w)×0.5 cm (h)) from bacterial cellulose membranes. Append a 10 cm cotton string to each corner of the bag to serve as tie string.

2. Preparation of filling materials: Prepare the filling by mixing propylene glycol, methyl cellulose, and distilled water according to weight ratio of 75:1:24 until homogeneous. Fill the mixture into the prepared rectangular bacterial cellulose ice bag and sterilize the bag at 120 degrees Celsius for 30 minutes. A control ice bag was also made from PVC materials. The extent of condensation, mechanical strength and temperature change during effective application period of the bags were measured and compared as shown in Table 1.

TABLE 1
Technical parameters of ice bags

| Parameters | PVC | | Bacterial cellulose | |
|---|---|---|---|---|
| Extent of | Rapid condensation at | | No condensation, | |
| condensation | the surface, resulting | | dry at the surface | |
| | in runny water | | | |
| | droplets | | | |
| Mechanical | 8.36 | GPa | 31.12 | GPa |
| strength | | | | |
| Temperature | | | | |
| change | 20° | C. | 13° | C. |

### EXAMPLE 2

### Preparation of Knee Ice Bags

1. Preparation of packing materials: Using the knee area as blueprint, make a rectangular ice bag (30 cm (l)×20 cm (w)×0.4 cm (h)) from bacterial cellulose membranes. Append a 15 cm cotton string to each corner of the bag to serve as tie string.

2. Preparation of filling materials: Prepare the filling by mixing medical alcohol, methyl cellulose and distilled water at weight ratio 70:1:29 until homogeneous. Fill the mixture into the prepared rectangular bacterial cellulose ice bag and sterilize the bag at 120 degrees Celsius for 30 minutes. A control ice bag was also made from PVC materials. The extent of condensation, mechanical strength and temperature change during effective application period of the bags were measured and compared as shown in Table 2.

TABLE 2
Technical parameters of ice bags

| Parameters | PVC | | Bacterial cellulose | |
|---|---|---|---|---|
| Extent of | Rapid condensation at the | | No condensation, dry | |
| condensation | surface, resulting in runny | | at the surface | |
| | water droplets | | | |
| Mechanical | 8.73 | GPa | 30.08 | GPa |
| strength | | | | |
| Temperature | 19° | C. | 12° | C. |
| change | | | | |

### Example 3

### Preparation of Ankle Ice Bags

1. Preparation of packing materials: Using the ankle area as blueprint, make a rectangular ice bag (18 cm (l)×10 cm (w)×0.5 cm (h)) from bacterial cellulose membranes. Append a 10 cm cotton string to each corner of the bag to serve as tie string.

2. Preparation of filling materials: Prepare the filling by mixing propylene glycol, medical alcohol, methyl cellulose, and distilled water at a weight ratio of 40:25:1:34 until homogeneous. Fill the mixture into the prepared rectangular bacterial cellulose ice bag and sterilize the bag at 120 degrees Celsius for 30 minutes. A control ice bag was also made from PVC materials. The extent of condensation, mechanical strength and temperature change during effective application period of the bags were measured and compared as shown in Table 3.

TABLE 3
Technical parameters of ice bags

| Parameters | PVC | | Bacterial cellulose | |
|---|---|---|---|---|
| Extent of | Rapid condensation at the | | No condensation, dry | |
| condensation | surface, resulting in runny | | at the surface | |
| | water droplets | | | |
| Mechanical | 8.91 | GPa | 30.88 | GPa |
| strength | | | | |
| Temperature | 20° | C. | 14° | C. |
| change | | | | |

In the above examples, the fermentation culture, dimension of the bacterial cellulose membrane, and the length of the tie string can be adjusted according to processing variables to control the mechanical strength and other properties of the bacterial cellulose ice bags.

It should be noted that the above examples are non-limiting embodiments of the present invention. Clearly there can be many variants to the above non-limiting examples. All variants directly or indirectly conceived by the skilled in the art are considered covered by the present invention.

1) A bacterial cellulose ice bag of which the packaging material is made of bacterial cellulose membranes.

2) The production method for the bacterial cellulose ice bag in claim 1 consists of the following steps:
a) Selection of a cellulose-producing microorganism as starting culture for the fermentation of bacterial cellulose membranes
b) Making an ice bag according the shape of the affected area with three sealed side and one open side from water-impermeable cellulose membranes (under normal room temperature and pressure) with appropriate mechanical strength
c) Filling the bag with filler materials and then sterilizing at 120 degrees C. for 30 minutes and sealing the bag aseptically.

3) The microorganism in claim 2 for the production of bacterial cellulose ice bag is Gluconacetobacter xylinum 323 strain.

4) The filling materials in claim 2 are cooling agent, a mixture of cooling agent and thickener, a mixture of cooling agent and distilled water, or a mixture of cooling agent, thickeners and distilled water.

5) The said cooling agent in claim 4 is glycerol, ethanol or a mixture of glycerol and ethanol. The said thickener is methyl cellulose.

6) The said thickener in claim 4 is methyl cellulose.
